(19) United States
(12) Patent Application Publication
(10) Pub. No.: US 2004/0086481 A1
(43) Pub. Date: May 6, 2004

(54) Cationic polymer-nucleic acid complexes and methods of making them

(76) Inventors: Martin Charles Garnett, Draycott (GB); Nicolas Andrew Jones, Bristol (GB); Benjamin James Rackstraw, Arundel (GB); Fabio Bignotti, Bedizzole (IT)

Correspondence Address: BOZICEVIC, FIELD & FRANCIS LLP, 200 MIDDLEFIELD RD, SUITE 200, MENLO PARK, CA, 94025, US

(21) Appl. No.: 10/240,877

(22) PCT Filed: Apr. 12, 2001
(86) PCT No.: PCT/GB01/01687

(30) Foreign Application Priority Data
Apr. 14, 2000 (GB) 0009201.5

(51) Int. Cl. A61K 48/00; C08G 63/48; C08G 63/91

(52) U.S. Cl. 424/78.26; 514/44; 525/54.2

(57) ABSTRACT

A nucleic acid complex for delivering a nucleic acid or a derivative thereof to a cell comprises the components: A. a nucleic acid or a derivative thereof; B. a cationic polymer; and C. a preformed polyethylene glycol-cationic polymer copolymer. The complex has a conformation in which the nucleic acid or derivative thereof is condensed and wherein component C is bound to component A such that the poylethylene glycol groups of component C are located at the surface of the complex. The complexes which are stable to aggregation are useful for the delivery of nucleic acids or derivatives thereof to cells in biological systems.

### INTRODUCTION

The present invention relates to cationic polymer-nucleic acid complexes which have use in the delivery of nucleic acid to cells in biological systems, for instance in gene therapy. The invention also relates to methods of making such complexes and to gene therapy using such complexes.

The control of all living processes is mediated through DNA. DNA encodes proteins which, as enzymes, hormones and other regulatory factors, carry out the processes which enable living organisms to function. DNA also encodes for regulatory sequences which control the expression of proteins.

Because of its central role in living organisms, DNA makes an ideal therapeutic target. It is thought that many diseases could be controlled by the manipulation of DNA in living organisms. A large number of diseases are due to altered or missing DNA sequences. These include diseases resulting from single gene defects, for example cystic fibrosis and Duchenne muscular dystrophy, which are incurable by conventional medicines. It is thought that such single gene defects could be cured if a functioning piece of DNA could be delivered to the critical cells in which the functioning of this gene is essential. Similarly, a range of other chronic diseases including cancer, diabetes, and viral infections might also be treatable by a similar approach. Also, the use of DNA vaccines, where gene transfer produces a protein to stimulate an immune response, is thought to have a role in disease prevention and cancer therapy.

The key factor limiting therapies based on DNA manipulation is the ability to deliver therapeutic nucleic acids to the nuclear compartment of the appropriate cells. DNA is a long fragile molecule which is highly negatively charged (one negative charge per phosphate group) and which is readily cleaved by nucleases present both in extracellular fluids and intracellular compartments. As a high molecular weight, highly charged molecule it will not cross the lipid membranes surrounding the cell, nor can it readily escape from endosomal compartments involved in the uptake of macromolecules into cells. Even oligonucleotides, although smaller in molecular weight and in some analogues uncharged, show significant problems of stability and uptake.

A number of different vectors have been proposed for gene therapy. Viruses are an obvious choice. Viruses cause disease by inserting DNA into cells and have, therefore, evolved to be effective in delivering DNA into cells. A number of different viruses have been developed for gene therapy by deletion of DNA so that they are no longer capable of replicating, and to create space in their structure to include exogenous DNA for delivery to cells. Examples of such constructs include retroviruses, Adenoviruses and Herpes simplex viruses.

All of these viral systems suffer from a number of delivery problems. Viruses are immunogenic, they may revert to wild type and, thus, become capable of causing disease. They are difficult to produce on a large scale, and quality control and quality assurance is difficult to achieve. Viral tropism needs to be modified to reflect the disease target. More significantly, the payload of DNA that viruses can carry is small.

Non-viral systems which can incorporate viral features facilitating DNA transfer into cells would be a preferable alternative for DNA therapies. They offer the prospect of lower immunogenicity, delivery of a larger DNA construct size, easier formulation and better quality control because all of the components can be purified and analysed separately. Also a similar delivery system could be used to carry pieces of DNA for different applications, instead of having to construct a new virus for each disease treatment.

There are two principal types of vector proposed for non-viral DNA delivery: cationic lipids and cationic polymer. Both of these types of construct use the positive charge in the molecule to at least partially neutralise the negative charge on the DNA to condense the DNA and to protect it from nucleases.

Cationic lipid formulations suffer from a number of shortcomings. The lipids used in these formulations are often toxic, and their use as delivery vehicles for nucleic acid to cells can be limited by the toxicity of this component. Cationic lipid formulations are also unstable and have a relatively short shelf life. The short shelf life is at least partly due to the tendency of these formulations to aggregate. Furthermore, lipid formulations are generally expensive due to the cost of the lipids.

The use of cationic polymers overcomes some, but not all, of the problems associated with cationic lipid formulations. Polycationic polymers are, however, generally cytotoxic although some cationic polymers with lower toxicity have been reported. Cationic polymers are generally cheap to produce, and do not have the shelf life problems associated with cationic lipids.

Cationic polymers are very efficient at condensing DNA into a small volume and at protecting DNA from degradation by serum nucleases. Under appropriate conditions, tightly packaged DNA is produced in the form of a toroid, in which the DNA is circumferentially wrapped. The interactions of cationic polymer with DNA are complicated and are explained by polyelectrolyte theory. Interaction is through an equilibrium reaction in which adjustment of the environmental conditions, (salt concentration, pH, molecular weight of each of the polymers) will affect the composition and form of the complexes. Generally, disproportionation and co-operative binding occur at charge ratios less than 1, adding to the complexity of the mixtures. Because of disproportionation, not all the complexes in a mixture will have the same composition.

In the formation of the toroids, the processes of condensation of DNA and aggregation of particles are competing, so that these systems tend to be unstable with time and form larger aggregates. This is influenced by the charge ratio of the complexes, and can be reduced by using an excess of one of the components. Generally such complexes are, therefore, made with an excess of polymer, although similar complexes with an excess of DNA also have some favourable properties.

DNA complexes made using cationic lipids or using cationic polymers suffer from a number of similar problems relating to their stability. The presence of serum tends to destabilise the complexes. The charge of the complexes causes problems in vivo, as these complexes are then readily recognised by the reticuloendothelial system (RES), which rapidly removes them-from the circulation before they reach their target site. A significant problem with both of these approaches is that they are not as effective at delivering DNA to cells as viruses. Although some formulations are more effective than others in delivering DNA the best cationic lipid and cationic polymer formulations reported in the prior art to date are still considerably less efficient than viral delivery systems.

The lack of efficiency of cationic polymer-DNA delivery systems may relate to the efficiency with which they can be taken up into cells, and with which they can escape from the endosomal compartment of the cell, into the cytoplasm and then to the nucleus. For this reason there has been much research into incorporating ligands and other biologically-active molecules which recognise cell surface receptors involved in endocytosis, and into the use of molecules, such as amphipathic peptides, which can disrupt endosomal membranes. However, even with the incorporation of these adjuvants, the efficiency of cationic polymer-DNA delivery systems is not as good as that of viral systems, although DNA-cationic polymer complexes in the presence of replication incompetent viruses do approach the efficiency of transfection seen by viruses.

It, therefore, seems likely that the size and stability of the complexes formed, and their tendency to aggregate, both in vitro and in vivo is a key factor involved in formulating non-viral DNA delivery systems which have a high transfection efficiency.

In colloidal drug delivery systems it has been recognised for many years that coating particles with a hydrophilic polymer, such as polyethylene glycol (PEG), can markedly improve performance by reducing interaction with serum proteins (opsonisation) and, therefore, uptake into the RES. This occurs due to the formation of a sterically-stabilised hydrophilic layer on the surface of the colloidal particle. The sterically-stabilised layer can also prevent aggregation by creating a barrier between the particles and preventing interaction between the surfaces of different particles. Colloidal drug delivery systems using this principle have been reported for both polymeric (nanoparticle) and lipidic (liposomal) delivery systems.

A number of groups have attempted to incorporate PEG onto the surface of their DNA delivery constructs to improve the properties of the DNA delivery systems. PEG conjugated to hydrophobic or lipidic groups has been reported to stabilise the lipid DNA complexes and prevent aggregation. However, even with these additions, the particle size of such complexes is reported to be around 400 nm. Intravenous injection of these particles into experimental animals has demonstrated that lung tissue is the principal tissue transfected with reporter genes incorporated into these complexes. These results suggest that, in vivo, the complexes are of a large particle size and are trapped in lung as the first capillary bed. While this process has resulted in an increased stability of these complexes to aggregation thus increasing their useful shelf life, the ability of these complexes to transfect cells has not been improved appreciably. Similarly, a number of groups have used PEG cationic polymer co-polymers (known as PEGylated cationic polymers) to produce DNA complexes. These are variously reported to stabilise the complexes, to produce smaller and more consistent particles, and to improve the resistance of the DNA in the complexes to digestion by nucleases. However, the stability of the complexes to serum nuclease is not very high, perhaps due to the fact that the complexes formed are less densely packed, due to the incorporation of PEG residues within the core of the DNA polymer complex.

A solution to this problem, proposed in WO 98/19710, involves the use of a two-step procedure in which the DNA is first condensed by a cationic polymer and then a hydrophilic polymer is covalently bonded to the cationic polymer after condensation of the DNA by the cationic polymer. This method has significant disadvantages in that the hydrophilic polymer can also react with the DNA thus reducing the availability of the DNA inside the cell. Because the complexes are not stabilised before the reaction can occur, the particle sizes and composition are not optimal before the reaction with the hydrophilic polymer takes place. Also the chemical reaction can result in the cross-linking of the complexes to produce a larger particle. Technically, this is a more difficult procedure than producing self-assembling particles by simple admixture of the components. Furthermore, after the reaction with the hydrophilic polymer excess polymer and other reagents must be removed from the complexes before use.

It is an object of the invention to overcome at least some of the above problems.

### STATEMENTS OF INVENTION

The present invention provides a nucleic acid complex which overcomes or ameliorates at least some of these and other problems associated with prior art complexes. Accordingly, the present invention provides a nucleic acid complex for delivering a nucleic acid or a derivative thereof to a cell which complex comprises the components:

A. a nucleic acid or a derivative thereof;

B. a cationic polymer; and

C. a preformed polyethylene glycol-cationic polymer co-polymer,

which complex has a conformation in which the nucleic acid or derivative thereof is condensed and wherein component C is bound to component A such that the polyethylene glycol groups of component C are located at the surface of the complex.

The nucleic acid complex of the invention overcomes various disadvantages associated with prior art complexes. For instance, the use of component B allows control of the PEG density on the surface of the complex, ensuring that excess PEG does not become incorporated into the central condensed core of the complex to interfere with the condensation of the nucleic acid. Unlike the invention described in WO 98/19710 where the complex is stabilised after its formation, the presence of the PEG at the surface of the complex of the present invention allows an optimal arrangement of the complex to occur during its formation. Also compared to the teaching in WO 98/19710, the present invention ensures that no PEG is introduced to a nucleic acid-cationic polymer complex in such a way that it could react with the nucleic acid or cause cross-linking of complexes to produce larger particles. A further advantage of the present invention is that optimal complexes are formed at low ratios of cationic polymer to DNA, so that all or most of the cationic polymer is incorporated into the complexes.

There is therefore little or no free cationic polymer present and toxicity due to the cationic polymer component is substantially reduced.

According to another aspect the present invention provides a method of making a nucleic acid complex which comprises contacting a nucleic acid or a derivative thereof with a cationic polymer and a preformed polyethylene glycol-cationic polymer co-polymer wherein the cationic polymer and the preformed polyethylene glycol-cationic polymer co-polymer contact the nucleic acid complex or a derivative thereof simultaneously or sequentially.

The nucleic acid or derivative thereof used as component A in the complex of the present invention may be DNA, RNA or an oligonucleotide. The nucleic acid may be an antisense nucleic acid.

The cationic polymer used as component B in the complex of the present invention is a polymeric material made up of a plurality of polycation molecules. The cationic polymer is, therefore, a polycationic material and has a size and structure capable of condensing the nucleic acid or derivative thereof and has a plurality of cationic groups which neutralise phosphate groups in the nucleic acid or derivative thereof. Examples of cationic polymers that may be used as component B include linear polyamidoamines, dendritic polyamidoamines, polyethylenimines, aminosugar polymers, polyaminoacids, peptoids and recombinant proteins. Component B may comprise mixtures of two or more of such polycations.

Linear polyamidoamines have a backbone comprising amido and amine groups. They are degradable in water since they contain hydrolysable amidic bonds in their main chain together with nucleophilic amine groups. They may be prepared by the reaction of aliphatic monoamines or diamines and bisacrylamides. WO 97/25067 describes the preparation of linear polyamidoamines suitable for use in the present invention and the contents of that document are incorporated herein by reference.

Dendritic polyamidoamines are highly branched polyamidoamines with branching occurring at the amino groups in the molecule. These dendrimers are soluble in water and have a high cationic charge density of primary amine groups on the polymer surface.

Polyethylenimines are polymers in which every third atom on the polymer backbone is a nitrogen atom. They may be linear or branched.

Aminosugar polymers have a glucose backbone with amino group-containing side chains,

Polyaminoacids are synthetic polymers of basic aminoacids. Examples include poly-L-lysine and poly-L-ornithine.

Polyethylene glycol-cationic polymer co-polymers, which are useful as component C in the complex of the present invention, are cationic polymers to which one or more polyethylene glycol compounds are attached. Such PEG-cationic polymer co-polymers, which can be block or graft co-polymers, may be prepared by providing the PEG molecule with reactive groups capable of reacting with reactive groups present on or provided on the cationic polymer and contacting the reactive PEG molecules with the reactive cationic polymer molecules under conditions such that the PEG molecule becomes attached or linked to the cationic polymer molecule by way of covalent bonding. Methods by which PEG molecules can be attached to or linked to cationic polymer molecules are known in the art. In this respect, reference is made to WO 99/01469 which describes a process for attaching a PEG compound to a macromolecule. The contents of this document are incorporated herein by reference.

The cationic polymer that may be used to form the PEG-cationic polymer co-polymer (component C) may be the same as, similar to or different from the cationic polymer used as component B in the complex of the present invention.

It is known that different cationic polymers bind nucleic acid differently. The strength of binding of the cationic polymer to the nucleic acid or derivative thereof is, therefore, an important consideration to be taken into account in choosing the cationic polymer to:

a) ensure a good compaction of the central core;

b) ensure a sufficient strength of binding to prevent or reduce nuclease attack on the nucleic acid or derivative thereof; and

c) ensure strong binding of the PEG-cationic polymer co-polymer to the surface of the condensed nucleic acid or derivative thereof.

Alternatively, the cationic polymer of components B and C may be chosen in order to ensure weaker or reversible bonding in order to allow intracellular release of the nucleic acid or derivative thereof from the complex.

Some cationic polymers, for instance polyethylenimines and polyamidoamine dendrimers are believed to assist the endosomal escape of DNA (i.e., they have good transfection activity in the absence of compounds which enhance endosomal escape, e.g., NH₄Cl, chloroquine, amphipathic peptides or viral components) and such cationic polymers are, therefore, preferred as component B in the complex of the invention.

Cationic polymers also differ in toxicity. Thus, the use of different cationic polymers together as component B or the use of a cationic polymer (component B) which is different from the cationic polymer in the PEG-cationic polymer co-polymer (component C) may desirably reduce the overall toxicity of the complex of the invention. However, this apparent advantage has to be weighed against the effect on the stability of the complex of using a cationic polymer for component B which is different chemically from the cationic polymer used in the PEG-cationic polymer co-polymer of component C if these cationic polymers have substantially different binding affinities with the nucleic acid or derivatives thereof. It is, therefore, preferred that the binding affinities of the cationic polymer of component B and of the cationic polymer in the PEG-cationic polymer co-polymer of component C are similar. This can be most easily achieved in cases where the cationic polymer segment of the PEG-cationic polymer co-polymer (component C) is the same as or is similar to the cationic polymer (component B).

According to a preferred embodiment, the cationic polymer (component B) and the cationic segment(s) of the PEG-cationic polymer co-polymer (component C) are linear polyamidoamine polymers as disclosed in WO 97/25067. The PEG-cationic polymer co-polymer is preferably a block co-polymer with the structure [poly(amidoamine)-(ethyleneglycol)_{y}]ₓ wherein x is from 1 to 50 and y is from 1 to 200 or a triblock ABA-type co-polymer with the structure (ethyleneglycol)_{y}-poly(amidoamine)-(ethyleneglycol)_{y} wherein each y is independently 1 to 200. Such PEGylated poly(amidoamine) block co-polymers and methods of making them are described in WO 97/25067 and the contents of this document are incorporated herein by reference.

According to a preferred embodiment, the number of charged groups in component B will be within the range of from 90 to 10% of the number of charged groups in component C. This allows control over the number of polyethylene glycol (PEG) groups located at the surface of the complex. More preferably, the number of charged groups in component B will be from 80 to 20% of the number of charged groups in component C.

According to a preferred embodiment the complex of the invention comprises at least one biological recognition unit to enhance binding, uptake by receptor mediated mechanisms, or for targeting to certain cell types of tissue. An appropriate biological recognition unit may either be a ligand, a molecule or a structure recognised by a cell surface receptor (e.g., a sugar, peptide or protein), or a molecule or structure capable of binding to a receptor or cell surface structure, e.g., an antibody, antibody fragment or lectin. The biological recognition unit should be incorporated into or attached to the surface of the nucleic acid complex which may interact with the cell or tissue. Such incorporation may be achieved, for instance, by attachment to the terminus of the PEG moiety of the PEG-cationic polymer co-polymer before or after the complex has been assembled.

Suitable recognition signals include ligands for binding and endocytosis especially of DNA delivery systems such as transferrin, for example see E. Wagner, M Cotton, R Foisner and M L Bernstiel (1991) Proc. Natl. Acad. Sci. USA 88, 4255-4259; carbohydrate residues, for example galactose, or mannose residues to target to hepatocytes or macrophages respectively. (G Ashwell and J Harford (1982) Ann. Rev. Biochem. 51, 531-54 describes carbohydrate specific receptors of the liver and use of asialoglycoprotein receptor in gene targeting with attachment of asialo-orosomucoid to PLL-DNA constructs is described in G Y Wu and C H Wu (1988) Biochemistry 27, 887-892); folate receptors as described in C P Leamon and P S Low (1991) Proc. Natl. Acad. Sci. USA 88, 5572-5576 and G Citro, C Szczylik, P Ginobbi, G Zupi and B Calabretta (1994) Br. J. Cancer 69, 463-464; monoclonal antibodies, especially those selective for a cell-surface antigen; and any other ligand which will mediate endocytosis of macromolecules.

Monoclonal antibodies which will bind to many of these cell surface antigens are already known but in any case, with today's techniques in relation to monoclonal antibody technology, antibodies can be prepared to most antigens. The antigen-binding portion may be a part of an antibody (for example a Fab fragment) or a synthetic antibody fragment (for example a single chain Fv fragment [ScFv]). Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982).

Chimaeric antibodies are discussed by Neuberger et al (1988, 8^{th} International Biotechnology Symposium Part 2, 792-799).

Suitably prepared non-human antibodies can be “humanized” in known ways, for example by inserting the CDR regions of mouse antibodies into the framework of human antibodies.

Other functionalities may also need to be incorporated into a nucleic acid delivery system. These may be, for example, to aid the escape of the nucleic acid from the endosomal compartment, or to enhance localisation of the nucleic acid to the nucleus. Both amphipathic peptides and viral components have been used in prior art systems to enhance endosomal escape. Suitable endosome disrupting agents such as viral fusogenic peptides and adenoviral particles have been described in J-P Bongartz, A-M Aubertin, P G Milhaud and B Lebleu (1994) Nucleic Acids Research 22, 4681-4688 and M Cotton, E Wagner, K Zatloukal, S Phillips, D T Curiel, M L Bernsteil (1992) Proc. Natl. Acad. Sci. USA 89, 6094-6098. Other peptides with different mechanisms of transporting proteins and oligonucleotides across cell membranes are known in the art. Such components may be coupled to the nucleic acid, coupled to the cationic polymer (component B), coupled to the cationic polymer segment of the PEG-cationic polymer co-polymer, or incorporated into the PEG-cationic polymer co-polymer at the PEG terminus, or at the PEG-cationic polymer junction. Alternatively they may be non-covalently incorporated into the complex either through ionic or hydrophobic interactions or via hydrogen bonding.

Both peptide and nucleic acid sequences are believed to favour nuclear localisation necessary for gene expression. Such sequences may be incorporated either through attachment to, or incorporation into, the nucleic acid complex.

The nucleic acid complex of the present invention may be made by contacting the nucleic acid or derivative thereof with components B and C, preferably in solution, more preferably in aqueous solution. In forming the complex, the order of addition of components and how the additions are made can influence the complex formed. There are a number of different ways in which a mixture of cationic polymer and PEG-cationic polymer co-polymer may be bound to the nucleic acid or derivative thereof. These are set out as follows:

1. addition of a preformed mixture of the cationic polymer (component B) and the PEG-cationic polymer co-polymer (component C) to the nucleic acid or derivative thereof;

2. addition of the nucleic acid or derivative thereof to a mixture of components B and C;

3. addition of component B to the nucleic acid or derivative thereof followed by the addition, to the mixture of component B and the nucleic acid or derivative thereof, of component C;

4. addition of the nucleic acid or derivative thereof to component B followed by the addition, to the mixture of component B and the nucleic acid or derivative thereof, of component C;

5. addition of component C to the nucleic acid or derivative thereof followed by the addition, to the mixture of component C and the nucleic acid or derivative thereof, of component B; and

6. addition of the nucleic acid or derivative thereof to component C followed by the addition, to the mixture of the nucleic acid or derivative thereof and component C, of component B.

We would expect the complexes formed from these three components to be thermodynamically stable. In this case it may be possible to mix the components in any order, followed by an equilibration process to allow the optimal complexes to form. However, at present, we find that the order of addition of components contributes significantly to creating the optimal complexes, and our preferred methods are in accordance with procedures 1 and 3 above. The complexes may be formed either using a slow or a rapid addition of components. A rapid addition of components, however, seems to give superior results.

According to a further aspect, the invention relating to nucleic acid complexes obtainable by a method of the invention.

According to a further aspect, the present invention provides the use of a nucleic acid complex of the invention in the manufacture of a medicament for the treatment of a disease of a living animal body including human.

A yet further aspect of the invention relates to a method of treating a disease of a living animal body, including a human, which disease is responsive to the delivery of nucleic acid or a derivative thereof to cells in the body which method comprises administering to the living animal body, including human, a therapeutically-effective amount of a nucleic acid complex of the invention as described herein.

Many diseases are known to result from the presence in the body of one or more defective genes. Examples of such genetic diseases include cystic fibrosis, Duchenne muscular dystrophy, haemophilia, phenylketonuria, thalassaemia and certain types of cancer. Reference is made to WO 97/25067 which contains a greater list of such diseases which are considered to be targets for gene therapy. The nucleic acid or derivative thereof which may be delivered to target cells according to the method described above using the nucleic acid complex of the invention may, according to one embodiment, be a gene or gene fragment which replaces the defective function of a defective gene in the target cells.

According to another embodiment, the nucleic acid or derivative thereof which may be delivered to the animal body, including human, by the use of the nucleic acid complex of the invention may be a nucleic acid vaccine.

A still further aspect of the invention relates to a pharmaceutical composition comprising a nucleic acid complex of the invention together with a pharmaceutically-acceptable carrier. Such compositions may be formulated according to methods known in the art. Typically, the composition will be formulated for administration to the patient parenterally using, as carrier, sterile water or saline although formulations for administration to the patient by other means may be possible. In this respect the contents of WO 97/25067 are incorporated herein by reference.

The present invention provides nucleic acid complexes that are more stable to aggregation. While this property is clearly advantageous in facilitating transfection in vivo, the complexes of the invention are also useful for transfection in vitro, particularly in systems where serum is a beneficial component in the transfection medium.

### EXAMPLES

Materials and Methods

I Preparation of Cationic Polymer

MBA-DMEDA (Methylene bis acrylamide-dimethylethylene diamine) co-polymer (NG49)

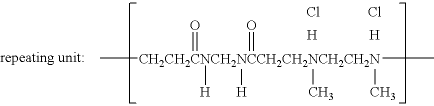

N,N′-dimethylethylenediamine (5.0 ml) was dissolved in distilled water (15 ml) in the presence of 4-methoxyphenol (20 mg). Methylene bisacrylamide (MBA) (7.17 g) was added and allowed to react at 25° C., under a nitrogen atmosphere and in the dark, for 2 days. Afterwards, the resulting solution was diluted, its pH was adjusted to pH 2 by the addition of aqueous HCl. It was, then, ultrafiltered in cold water through a membrane with M_{w} cut off 3000 and freeze-dried. Yield=11.21 g. Intrinsic viscosity at 30° C. in Tris buffer pH 8.09=0.48 dl/g. Weight-average molecular weight (M_{w}) determined by GPC=28200. Number-average molecular weight (Mₙ) determined by GPC=19800. H1-NMR (in D₂O, chemical shift in ppm with respect to TSP): δ=2.21 (s.6H, N—CH₃), 2.42 (t, 4H,CO—CH₂), 2.53 (t,4H,CO—CH₂—CH₂—N),2.71 (t,4H, N—CH₂—CH₂—N), 4,55 (m,2H, N—CH₂—N).

Gel Permeation Chromatographic analysis was performed using TSK-GEL G3000 PW and TSK-GEL G4000 PW columns connected in series, using TRIS buffer pH 8.09 as mobile phase (flow: 1 ml/min) and a UV detector operating at 230 nm. From the GPC chromatograms the number average molecular weight (Mₙ) and the weight average molecular weight (M_{w}) were calculated.

Intrinsic viscosity measurements were measured at 30° C. in TRIS buffer pH 8.09 by means of Ubbelohde viscometers.

II Preparation of PEG-poly(amidoamine)-PEG triblock copolymer (NG47)

A. Preparation of monomethoxy-PEG piperazinyl formate (MPEG-PF).

Monomethoxy-PEG 1900 (30.15 g) was dissolved in “alcohol-free” CHCl₃ (160 ml). The solution was dried overnight over calcium hydride, which was then separated by filtration. Afterwards, 1,1′-carbonyldiimidazole with a degree of purity of 97% (3.98 g) was added and the solution was allowed to stand at 30° C. for 30 min. Cold water was added (40 ml) and the mixture was stirred for 10 min. After separation of the phases, N,N′-dimethylethylenediamine (2.6 ml) was added to the organic phase and allowed to react for 20 hours at 25° C. The solution was diluted with CHCl₃ (400 ml), extracted with water (5×120 ml), dried with Na₂SO₄, filtrated, concentrated in vacuo up to 150 ml and poured into diethyl ether. The precipitate was collected by filtration and dried to constant weight at 0.1 torr. Yield=24.5 g. The molecular weight, determined by potentiometric titration with 0.1 M HCl, was 2230.

B) Preparation of vinyl-terminated Poly(amidoamine) (VT-PAA).

N,N′-dimethylethylenediamine (5.53 g) was dissolved in distilled water (15 ml) in the presence of 4-methoxyphenol (20 mg); MBA (10.07 g) was added and allowed to react at 20° C., under a nitrogen atmosphere and in the dark, for 24 hours. Afterwards, the resulting solution was diluted, ultrafiltered in cold water through a membrane with M_{w} cut off 10,000 and freeze-dried. Yield=3.80 g. Intrinsic viscosity at 30° C. in Tris buffer pH 8.09=0.22 dl/g. Mₙ determined by GPC=7200.

C) Preparation of PEG-Poly(amidoamine)-PEG tri-block Copolymer

VT-PAA (2.72 g) of step b) was dissolved in distilled water (5 ml), then MPEG-PF of step a) (1.87 g) was added and the reaction mixture was allowed to stand at 25° C. for 24 hours under a nitrogen atmosphere and in the dark. The resulting solution was diluted, its pH was adjusted to pH 2 by the addition of aqueous HCl, then it was ultrafiltered in cold water through a membrane with M_{w} cut off 50,000 and freeze-dried. Yield=4.26 g. H1-NMR (in D₂O, chemical shift in ppm with respect to TSP): δ=2.21 (s, N—CH₃), 2.42 (t, CO—CH₂), 2.53 (t,CO—CH₂—CH₂—N), 2.71 (t, N—CH₂—CH₂—N), 3.76 (broad s, O—CH₂,—CH₂), 4.55 (m, N—CH₂—N). PAA content estimated from the intensity ratio of the PEG signal (3.76 ppm) to the PAA signals (2.21, 2.42, 2.53 and 2.71 ppm)=65 wt %. Intrinsic viscosity at 30° C. in Tris buffer pH 8.09=0.26 dl/g. The GPC trace exhibits one peak displaced towards lower retention times compared to both VT-AA and MPEG-PF.

III Polymer Blend Solutions

A 50/50 blend of NG49 and NG47 was prepared so that an equal molar proportion of cationic monomers of each was present. Stock solutions (10 mg/ml) of NG47 and NG49 in distilled water were prepared. Polymer mixtures were prepared by adding volumes of polymer stock solutions containing appropriate amounts of poly(amidoamine) (PAA) repeating units to give a total repeating unit concentration of 10 mg/ml. (The molecular weight of the NG49 repeating unit was 315 and the equivalent molecular weight of NG47 was therefore calculated as 424. Hence 1 mol of MBA DMEDA repeating units in NG49 would be contained in 315 g, while 1 mol of MBA

DMEDA repeating units with associated PEG in NG47 would weigh 424 g.)

To give an example of the preparation of a polymer solution: for a 50/50 mixture, NG49 (500 μl) was mixed with NG47 (673 μl)*, to give a total polymer concentration of 10 mgml⁻¹, and this was further diluted to give a working solution with total polymer concentration of 1 mgml⁻¹ (RU concentration 1 mg/1.1133 ml). Other polymer blends containing different proportions of each polymer were prepared along the same lines. * amount of NG47=(monomer Mwt of NG47/monomer Mwt of NG49)×amount of NG49 i.e., (424/315)×5=6.73, contained in 673 μl of 10 mgml⁻¹ solution.

IV Preparation of Nucleic Acid Complexes

Nucleic acid complexes were formed using, as nucleic acid,

a) The 6 kb plasmid pRSVluc, containing the firefly luciferase gene, obtained from Cobra Therapeutics (Keele, UK) and produced in bulk by Aldevron Inc. or

b) The 4.6 kb plasmid pCT0129L, containing the chloramphenicol acetyl transferase gene, obtained from GeneMedicine Inc (Houston, Tex., USA).

Both plasmids were supplied as 1 mgml⁻¹ working solutions in double distilled water and were used without further modification.

The amount of polymer blend solution required to form complexes with a certain total polymer:DNA ratio with a given quantity of DNA were calculated. For example to form complexes with polymer:DNA ratio of 2:1, using the NG49/47 50/50 blend, and 5 μg of DNA:

Amount of polymer blend=(RU M_{w} of NG49/monomer M_{w} of DNA)×amount of DNA×desired total polymer:DNA ratio×polymer RU concentration.

i.e., ((315/308)×5×2×1.133=11.88˜12 μl of 1 mgml⁻¹ polymer blend solution.

Experimental Section

A. Particle Size Determination

Cationic polymer-nucleic acid complexes were prepared as described above over a range of polymer:DNA ratios wherein the polymer comprised NG49 or blends of NG49 and NG47 as follows:

| | | |
|---|---|---|
| (i) | NG49/NG47 | (50/50) |
| (ii) | NG49/NG47 | (66/33) |
| (iii) | NG49/NG47 | (75/25) |

The complexes were assessed using photon correlation spectroscopy (PCS). PCS is a technique by which particle sizes can be determined by dynamic light scattering measured at 90° to the incident light. The technique provides the mean particle diameter and a measure of the polydispersity of the particles. It also counts the numbers of particles in the light beam.

Buffers were routinely filtered through a 0.22 μm filter prior to preparation of solutions for PCS. Polymer mixture was added to pCT0129L plasmid (10 μgml⁻¹) in 500 μl PBS and briefly vortex-mixed. Z-average particle sizes, polydispersity and count rate measurements were determined using a Malvern PCS4700 (Malvern Instruments), at 25° C., with a fixed angle of 90°, aperture size between 150 and 300 μm and laser power of 40 mW.

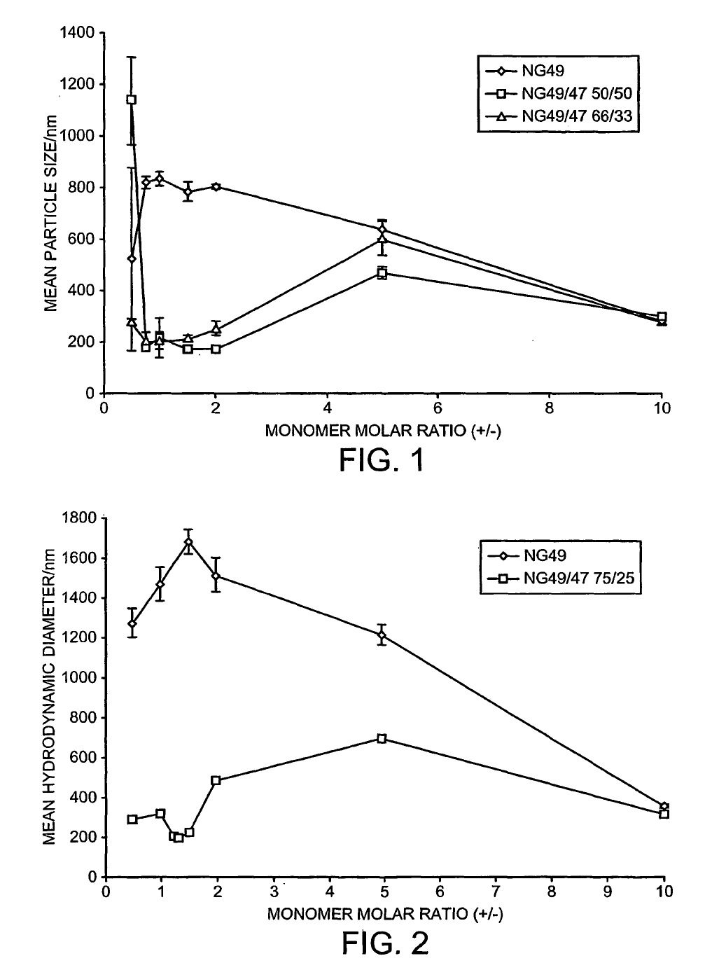

Plots of the mean particle size (nm) of the complexes against monomer molar ratio (i.e., polymer:DNA) for complexes formed between the DNA and the polymer using, as polymer, NG49 on its own, a 50/50 blend of NG49/NG47 and a 66/33 blend of NG49/NG47, are shown in FIG. 1. This figure shows that, at the optimal polymer:DNA ratios for the mixed systems, the mean particle size was significantly smaller than could be achieved by using NG49 on its own. The smaller sizes indicated that no aggregation was occurring at polymer:DNA ratios where NG49 is prone to aggregation. At higher polymer:DNA ratios the particle sizes of the complexes produced using mixed systems approach those shown for NG49, used on its own. Here it is assumed that at these ratios the NG49 is displacing NG47 from the complex.

A plot of the mean particle size (nm) of the complex formed using a mixed polymer system comprising 75/25 NG49/NG47 against polymer/DNA ratio is shown in FIG. 2. This shows that, at the optimal polymer:DNA ratio the mean particle size is much smaller than could be achieved using NG49 on its own.

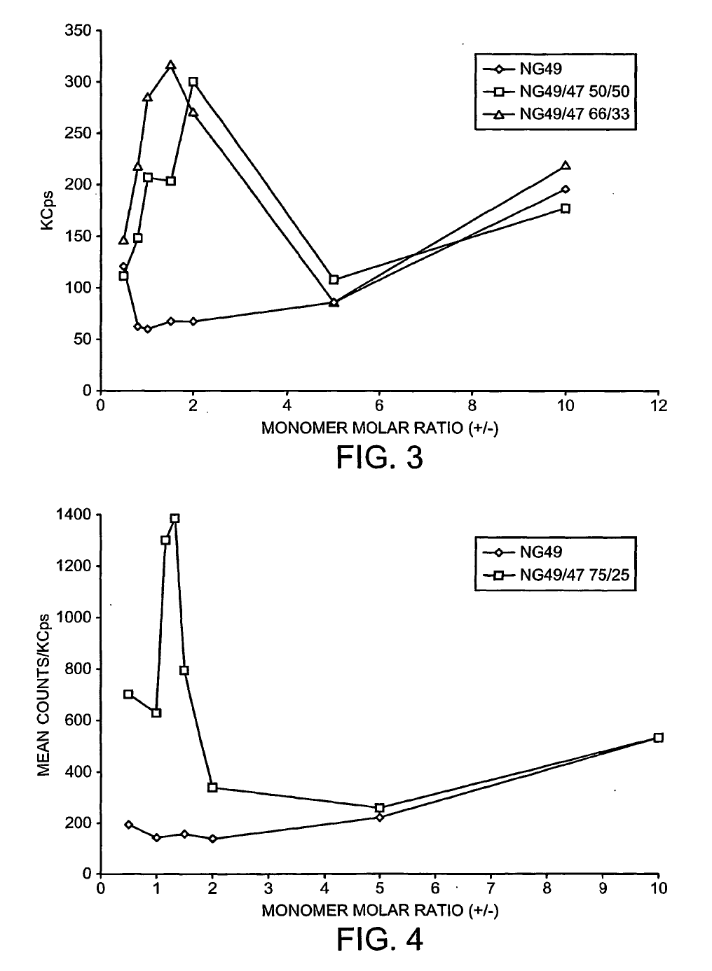

FIG. 3 shows the count rate measurements of the polymer:DNA complexes using, as polymer, NG49 on its own, a 50/50 blend of NG49/NG47 and a 66/33 blend of NG491NG47. FIG. 4 shows the count rate measurement for the mixed polymer system 75/25 NG49/NG47. In these Figures the count rate (counts×1000 per second) is plotted against the polymer:DNA ratio. High counts rates at low ratios for the mixed systems indicate that a larger number of particles are produced which are not aggregating. The low count rates shown for the complexes formed using NG49 on its own, at similarly low polymer:DNA ratios reflect a less efficient formation of particles. Again, it can be seen that at higher polymer:DNA ratios the mixed complexes behave like the NG49 only systems.

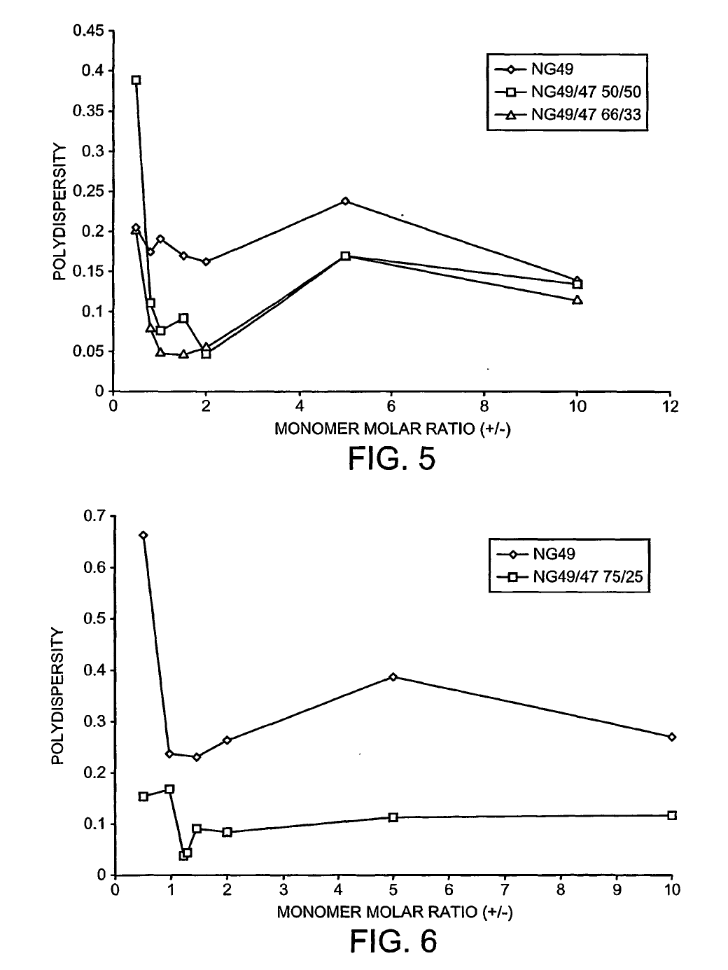

Plots of the polydispersity of the complexes against polymer:DNA ratios are shown in FIGS. 5 and 6. Low polydispersity is indicative of a uniform population with regard to particle size. A polydispersity below about 0.1 is generally regarded as monodisperse and is achieved here by the mixed systems 50/50 NG49/NG47, 66/33 NG49/NG47 (FIG. 5) and 75/25 NG49/NG47 (FIG. 6).

From the results shown in FIGS. 1 to 6 it can be seen that the optimum polymer: DNA ratio and characterisation of the particles produced using mixed polymer systems produces measurable differences with the different blend ratios of NG49/NG47. The particle sizes and polydispersity of these stabilised particles remained steady over a long period, with no sign of aggregation. One of the most significant aspects of the results reported above is that the optimal ratio of polymer to DNA to obtain stable particles is much lower using mixtures of cationic polymer and PEG-cationic polymer co-polymer (typically at ratios below 2:1) than for the system using the cationic polymer alone (5:1 ratio was required for NG49). It is known that cationic polymers effective in mediating transfection are often associated with toxicity to cells. The use of the mixed polymer system according to the present invention allows the amount of potentially toxic cationic polymer in a formulation for transfection to be reduced. In this respect, the present invention will clearly have favourable consequences for experiments in biological settings, such as transfection assays in cell culture and in vivo experiments.

B. Particle Morphology

Transmission electron micrographs of polymer-DNA complexes using different polymer systems to complex with the DNA were made at optimal polymer:DNA ratios.

Polymer mixture was added to pRSVLUC plasmid (10 μgml⁻¹) in tenfold diluted PBS. Samples prepared on coated copper grids, positively stained with uranyl acetate.

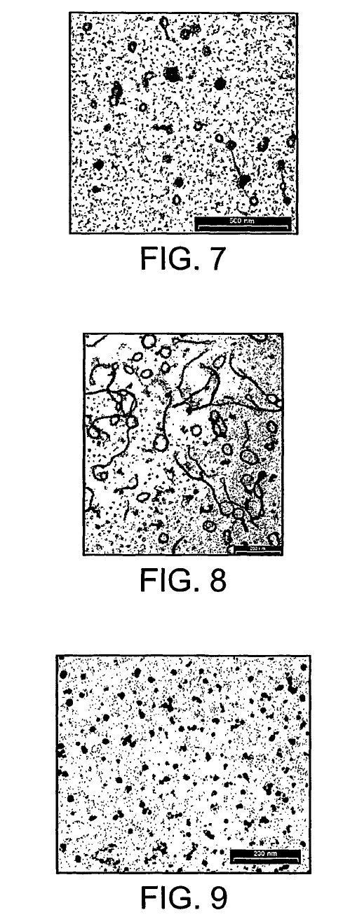

These are shown in FIGS. 7 to 9 wherein:

FIG. 7 is the TEM of a complex formed between NG49 and the DNA at a ratio of 5:1;

FIG. 8 is the TEM of a complex formed between NG47 and the DNA at a ratio of 1:1; and

FIG. 9 is the TEM of a complex formed between the polymer and the DNA wherein the polymer is a mixed system comprising 50/50 NG49/NG47 at a polymer:DNA ratio of 2:1.

A comparison of these Figures reveals that the complex formed from a mixed polymer system has a different morphology compared to the complexes formed using one component of the mixed system alone. The mixed system gave rise to seemingly spherical particles, rather than the toroidal structures normally seen for cationic polymer-DNA complexes. These spherical particles were shown to have a uniform, small size and were free from aggregation. The size and morphology of the particles of the mixed polymer system complex indicate that PEG is not being incorporated in the central condensed core.

C. Nuclease Protection

The DNase I digestion of the polymer-DNA complexes was studied according to the following procedure.

Polymer mixture was added to pCT0129L (10 μgml⁻¹) in PBS containing 5 mM MgCl₂. DNase I solution (1 μl of 10 μgml⁻¹) was added and UV absorbance at 260 nm measured, whilst incubating at 37° C. The DNase 1 had an enzyme activity of 4.4 activity units (Kunitz units) per microlitre.

Samples of DNA and polymer-DNA complexes were incubated in a spectrometer cell in the presence of and in the absence of the nuclease enzyme DNase I and absorbance at 260 nm was monitored over a period of 2000 seconds.

Absorbance of light having a wavelength of 260 nm by DNA is due to the nucleotide bases. When these are linked into a DNA strand some quenching occurs as a result of the stacking of the bases. This is most pronounced in double helical DNA. Degradation of the DNA reduces these stacking interactions and, as a result, the strength of absorbance at 260 nm increases. In this experiment the DNA used was pCT and the samples assayed were as follows:

Sample No. 1 DNA+DNase I

Sample No. 2 DNA+NG49(1:1)

Sample No. 3 DNA+NG49(1:1)+DNase I

Sample No. 4 DNA+NG47(1:2)

Sample No. 5 DNA+NG47(1:2)+DNase I

Sample No. 6 DNA+NG49+NG47(1:1:1)

Sample No. 7 DNA+NG49+NG47(1:1:1)+DNase I

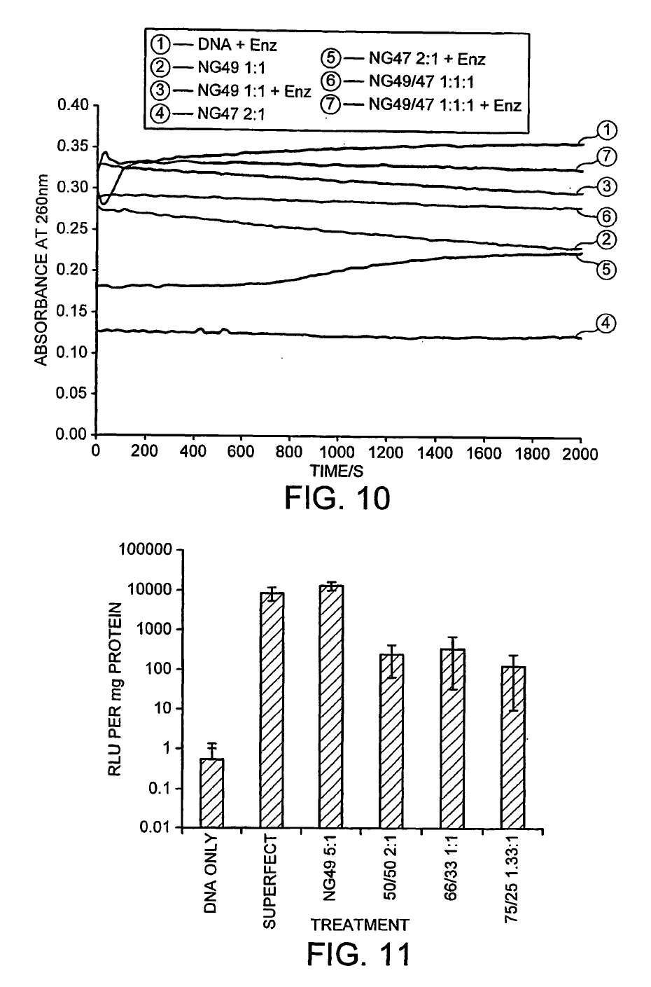

The plots of absorbance at 260 nm against time (s) are shown in FIG. 10.

It can be seen in FIG. 10 that the absorbance associated with the degradation of the plasmid DNA by the nuclease enzyme rises for the sample of the DNA alone and for the sample of DNA complexed with NG47 alone. In the latter case it is believed that the PEG molecules in NG47 interfere with the condensation of the DNA thus resulting in a more open complex susceptible to nuclease attack. NG49, which is cationic polymer containing no PEG molecules, condenses the DNA more effectively to give a polymer-DNA complex which is more resistance to nuclease attack as can be seen by the little apparent rise in absorbance caused by the presence of DNase I in sample 3.

The protection of the DNA from degradation by the nuclease enzyme offered by the mixed polymer system was seen to be similar to that conferred by the cationic polymer alone and not compromised by the inclusion of PEG in the system. Hence protection of the DNA against nuclease degradation by the mixed polymer system was superior to that afforded by the PEG-cationic polymer co-polymer when used alone. This indicates that the distribution of the PEG in the complex formed from the DNA, the cationic polymer and the PEG-cationic polymer co-polymer is not interfering with the condensation of the DNA.

D. Transfection of Cultured Cells

A549 cells were routinely grown in RPMI 1640 medium supplemented with heat inactivated foetal bovine serum. Cells were passaged twice weekly at 1:10 splitting ratio.

Wells were seeded with 10⁵ cells, grown for 24 hours. Complexes were prepared in OptiMEM I buffer (1 ml) by addition of polymer to pRSVLUC plasmid. These were incubated with cells for 4 hours, prior to replacement with fresh RPMI medium, and incubated for a further 48 hours. Subsequently, the incubating medium was removed and the cells were lysed using a composition (Promega) containing 125 mM Tris, pH 7.8 with phosphoric acid, 10 mM EDTA, 10 mM dithiothreitol (DTT), 50% glycerol and 5% triton X-100 (composition was used according to the manufacturers instructions). The lysate was collected and analysed.

Luciferase activity was measured using a Luciferase Assay System (Promega) and luminometer, according to provided protocol. A Bradford assay was performed to determine cellular protein, using Bradford Reagent from Sigma, and following the protocol provided.

The results are shown in FIG. 11.

The “DNA only” sample contained no polymer. The “superfect” sample comprised a dendrimer polymer obtained from Qiagen under the trade name SUPERFECT™. NG495:1 is a non-pegylated complex which is described (under the abbreviation NG30) in Biochemica et Biophysica Acta, 1517 (2000) 1-18. The remaining samples of FIG. 11 comprise polymer complexes according to the invention.

The invention is not limited to the embodiments hereinbefore described which may be varied without departing from the spirit of the invention.

1. A nucleic acid complex for delivering a nucleic acid or a derivative thereof to a cell which complex comprises the components:
A. a nucleic acid or a derivative thereof;
B. a cationic polymer; and
C. a preformed polyethylene glycol-cationic polymer co-polymer, which complex has a conformation in which the nucleic acid or derivative thereof is condensed and wherein component c is bound to component a such that the polyethylene glycol groups of component c are located at the surface of the complex.

2. A nucleic acid complex according to claim 1, wherein the number of charged groups in component B is in the range of from 90 to 10% of the number of charged groups in component C.

3. A nucleic acid complex according to claim 2, wherein the number of charged groups in component B is in the range of from 80 to 20% of the number of charged groups in component C.

4. A nucleic acid complex according to any one of claims 1 to 3, wherein the cationic polymer of component B is selected from linear polyamidoamines, dendritic polyamidoamines, polyethylenimines, aminosugar polymers, polyaminoacids, peptoids, recombinant proteins and mixtures of two or more thereof.

5. A nucleic acid complex according to any one of claims 1 to 4, wherein the polyethylene glycol-cationic polymer co-polymer of component C is selected from co-polymers of polyethylene glycol with a cationic polymer selected from linear polyamidoamines, dendritic polyamindoamines, polyethyleneimines, aminosugar polymers, polyaminoacids, peptoids recombinant proteins and mixtures of two or more thereof.

6. A nucleic acid complex according to any one of claims 1 to 5, wherein the polyethylene glycol-cationic polymer co-polymer of component C is selected from block co-polymers, multi-block co-polymers and comb-type co-polymers.

7. A nucleic acid complex according to any one of claims 1 to 6, wherein the cationic polymer of component B and the cationic polymer of the polyethylene glycol-cationic polymer co-polymer of component C have repeating units of the same chemical structure.

8. A nucleic acid complex according to claim 7, wherein both the cationic polymer of component B and the cationic polymer of the polyethylene glycol-cationic polymer co-polymer of component C are linear polyamidoamines.

9. A nucleic acid complex according to any one of claims 1 to 8, which has an electronic charge which is substantially neutral.

10. A nucleic acid complex according to any one of claims 1 to 9, wherein the ratio of the sum of cationic groups in components B and C to phosphate groups in component A is x:1, wherein x is a number less than 5.

11. A nucleic acid complex according to claim 10, wherein the ratio is x:1, wherein x is a number less than 2.

12. A nucleic acid complex according to any one of claims 1 to 11, which additional comprises at least one biological recognition signal entity bound to one of components A, B or C.

13. A nucleic acid complex according to any one of claims 1 to 12, wherein component A is DNA or a derivative thereof.

14. A nucleic acid complex according to any one of claims 1 to 12, wherein component A is RNA or a derivative thereof.

15. A nucleic acid complex according to any one of claims 1 to 14, which additionally comprises one or more components to enhance intracellular trafficking of the nucleic acid or the nucleic acid complex.

16. A method of making the nucleic acid complex, which complex is used to deliver a nucleic acid or a derivative thereof to a cell, and Which complex comprises the components:
A. a nucleic acid or a derivative thereof;
B. a cationic polymer; and
C. a preformed polyethylene glycol-cationic polymer co-polymer, which method comprises the step of contacting component A simultaneously or sequentially with component B and component C.

17. A method as claimed in claim 16, wherein a mixture of component B and component C is added to component A.

18. A method as claimed in claim 16, wherein component B is added to component A and then component C is added to the mixture of component A and component B.

19. A nucleic acid complex obtainable by a method of any of claims 16 to 18.

20. A nucleic acid complex according to any of claims 1 to 15 or 19 for use as a medicament.

21. The use of a nucleic acid complex according to any one of claims 1 to 15 or 19 in the manufacture of a medicament for treatment of a disease.

22. A pharmaceutical composition comprising a nucleic acid complex according to any one of claims 1 to 15 or 19 and a pharmaceutically-effective carrier.

23. A method of delivering a nucleic acid to a host comprising administering to the host a pharmaceutical composition according to claim 22.

24. A method according to claim 23 wherein the host is an animal body, including a human body, in need of treatment which comprises treating the animal body with a pharmaceutically effective amount of the pharmaceutical composition.
